(19) United States
(12) Patent Application Publication
(10) Pub. No.: US 2007/0259917 A1
(43) Pub. Date: Nov. 8, 2007

(54) Processes for the synthesis of 3-isobutylglutaric acid

(76) Inventors: Vinod Kumar Kansal, Haryana (IN); Brijnath P. Chaurasia, Uttar Pradesh (IN); Hitesh K. Patel, Gujarat (IN); Shivaji Haribhau Shelke, Maharashtra (IN); Yogesh P. More, Maharashtra (IN)

Correspondence Address: KENYON & KENYON LLP, ONE BROADWAY, NEW YORK, NY, 10004, US

(21) Appl. No.: 11/789,662

(22) Filed: Apr. 24, 2007

Related U.S. Application Data
(60) Provisional application No. 60/794,818, filed on Apr. 24, 2006.
(60) Provisional application No. 60/802,620, filed on May 22, 2006.

(51) Int. Cl. A61K 31/445; C07D 211/54; C07D 323/04

(52) U.S. Cl. 514/327; 546/216; 549/274

(57) ABSTRACT

Provided are processes for the synthesis of 3-isobutylglutaric acid, an intermediate in the synthesis of (S)-Pregabalin.

### CROSS-REFERENCE TO RELATED APPLICATIONS

This application claims the benefit of priority to U.S. provisional Application Ser. Nos. 60/794,818, filed Apr. 24, 2006 and 60/802,620, filed May 22, 2006, hereby incorporated by reference.

### FIELD OF THE INVENTION

The invention encompasses processes for the synthesis of 3-isobutylglutaric acid, an intermediate in the synthesis of (S)-Pregabalin.

### BACKGROUND OF THE INVENTION

(S)-Pregabalin, (S)-(+)-3-(aminomethyl)-5-methylhexanoic acid, a compound having the chemical structure, 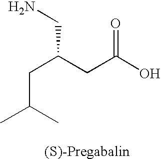
is a γ-amino butyric acid or (S)-3-isobutyl (GABA) analogue. (S)-Pregabalin has been found to activate GAD (L-glutamic acid decarboxylase). (S)-Pregabalin has a dose dependent protective effect on-seizure, and is a CNS-active compound. (S)-Pregabalin is useful in anticonvulsant therapy, due to its activation of GAD, promoting the production of GABA, one of the brain's major inhibitory neurotransmitters, which is released at 30 percent of the brains synapses. (S)-Pregabalin has analgesic, anticonvulsant, and anxiolytic activity.

(S)-Pregabalin is marketed under the name LYRICA® by Pfizer, Inc. in tablets of 25, 50, 75, 150, 200, and 300 mg doses.

(S)-Pregabalin may be prepared by converting 3-isobutylglutaric acid to 3-isobutylglutaric anhydride, followed by amidation to obtain the corresponding 3-(carbamoylmethyl)-5-methylhexanoic acid (referred to as CMH). The CMH is then resolved optically to give (R)—CMH, which is converted by a Hoffman-reaction to (S)-Pregabalin. See L. Martin, et al., “Pregabalin. Antiepileptic,” Drugs of the Future, 24(8): 862-870 (1999); U.S. Pat. No. 5,616,793. This process may be illustrated by the following Scheme 1.

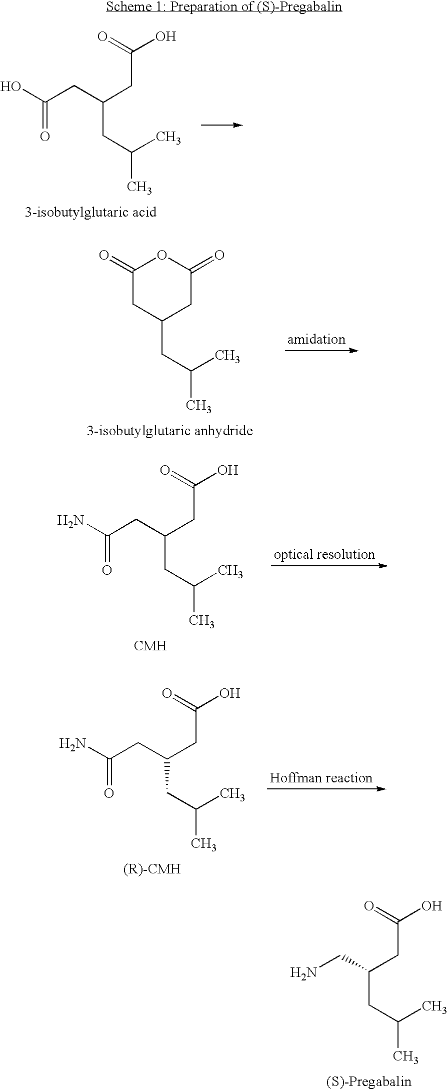

The 3-isobutylglutaric acid may be prepared by the condensation of isovaleraldehyde and ethylcyanoacetate, followed by a Michael addition, and hydrolysis. See Day and Thorpe, J. Chem. Soc., 117:1465 (1920); J. Casson, et al., “Branched-Chain Fatty Acids. XXVII. Further Study of the Dependence of Rate of Amide Hydrolysis on Substitution near the Amide Group. Relative Rates of Hydrolysis of Nitrile to Amide and Amide to Acid,” J. Org. Chem., 18(9): 1129-1136 (1953); P. D. Theisen, et al., “Prochiral Recognition in the Reaction of 3-Substituted Glutaric Anhydrides with Chiral Secondary Alcohols,” J. Org. Chem., 58(1): 142-146 (1993); M. S. Hoekstra, et al., “Chemical Development of CI-1008, an Enantiomerically Pure Anticonvulsant,” Organic Process Research & Development, 1(1): 26-38 (1997). This process may be illustrated by the following Scheme 2.

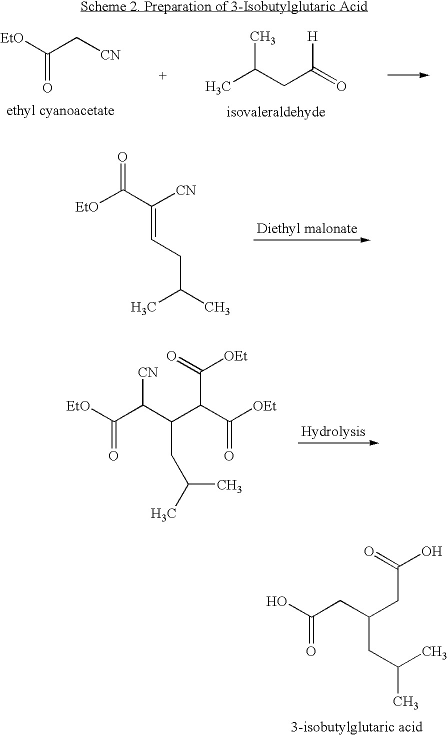

This process is also disclosed in U.S. Pat. No. 5,616,793 (“'793 patent”) and its corresponding International Publication WO 96/38405 (“WO '405”). The '793 patent and WO '405 disclose that the hydrolysis is complete after approximately 72 hours. '793 patent, col. 6, 11. 30-32; WO '405, p. 11, 11. 17-19.

Hence, there is a need in the art for processes for preparing 3-isobutylglutaric acid that can be performed in shorter time periods than those of the above-described prior art.

### SUMMARY OF THE INVENTION

In one embodiment, the invention encompasses a process for preparing 3-isobutylglutaric acid comprising: (a) combining isovaleraldehyde, a compound of the following formula II, 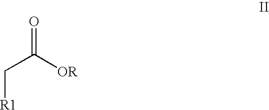
a compound of the following formula III, 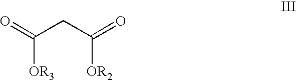
or a compound of the following formula IV, 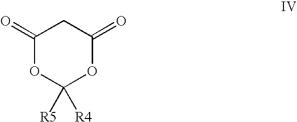
a non-polar organic solvent, and a first base to obtain a compound of the following formula V, 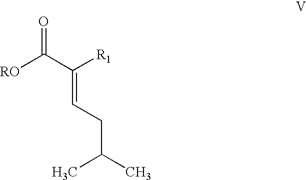
a compound of the following formula VIII, 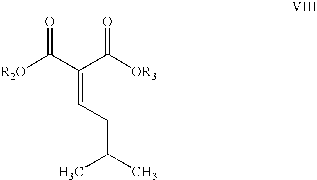
or a compound of the following formula X, 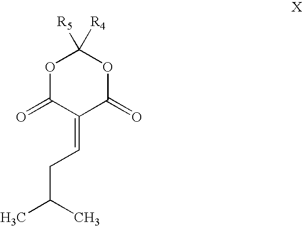
respectively; (b) combining a compound of formula IV with the compound of formula V, the compound of formula VIII, or the compound of formula X, a polar aprotic organic solvent, and a second base to obtain a compound of the following formula VII, 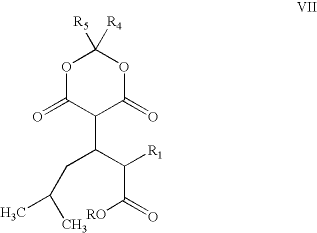
a compound of the following formula IX, 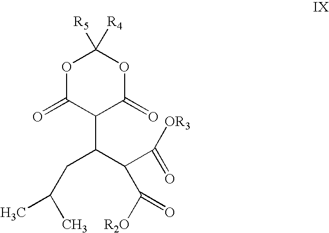
or a compound of the following formula XII, 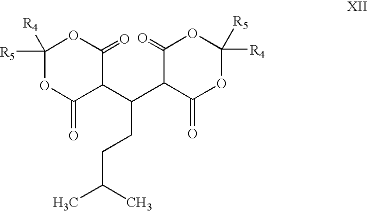
respectively; and (c) hydrolyzing the compound of formula VII, the compound of formula IX, or the compound of formula XII to obtain 3-isobutylglutaric acid, wherein R is H, linear or branched C₁₋₈ alkyl, or C₆₋₁₄ aryl; R₁ is H, CN, COOH, COO C₁₋₈ alkyl, COOC₆₋₁₄ aryl, or (R₆O)₂P═O; R₂ and R₃ are independently H, linear or branched C₁₋₈ alkyl, or C₆₋₁₄ aryl; R₄ and R₅ are independently H, linear or branched C₁₋₈ alkyl, or C₆₋₁₄ aryl; and R₆ is linear or branched C₁₋₈ alkyl or C₆₋₁₄ aryl.

In another embodiment, the invention encompasses a process for preparing 3-isobutylglutaric acid comprising: (a) combining isovaleraldehyde, a compound of the following formula III, 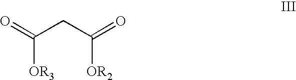
a non-polar organic solvent, an organic acid, and an organic base to obtain a compound of the following formula VIII; 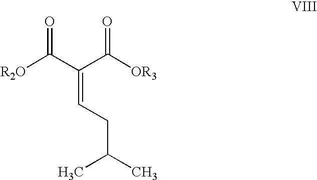
(b) combining the compound of formula VIII with the compound of formula III, a polar aprotic organic solvent, and an inorganic base to obtain a compound of the following formula XIII; and 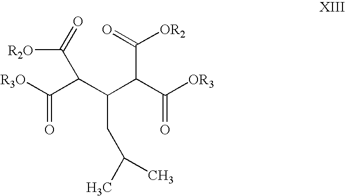
(c) hydrolyzing the compound of formula XIII to obtain 3-isobutylglutaric acid, wherein R₂ and R₃ are independently H, linear or branched C₁₋₈ alkyl, or C₆₋₄ aryl.

In another embodiment, the invention encompasses a process for preparing 3-isobutylglutaric acid comprising: (a) combining isovaleraldehyde, a compound of the following formula III, 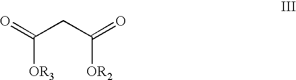
an alcohol, ammonium acetate and ammonia to obtain a compound of the following formula XIV; 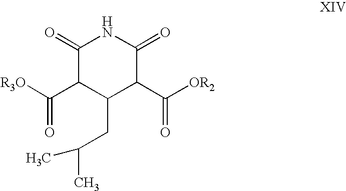
and (b) hydrolyzing the compound of formula XIV to obtain 3-isobutylglutaric acid, wherein R₂ and R₃ are independently H, linear or branched C₁₋₈ alkyl, or C₆₋₁₄ aryl.

In another embodiment, the invention encompasses a process for preparing 3-isobutylglutaric acid comprising: (a) combining isovaleraldehyde, a compound of the following formula II, 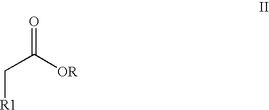
a non-polar organic solvent, and a first base to obtain a compound of the following formula V; 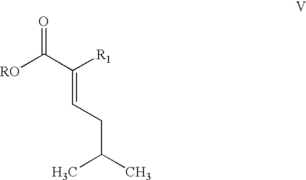
(b) combining the compound of formula V with a compound of the following formula III 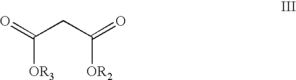
and a second base to obtain a compound of the following formula VI 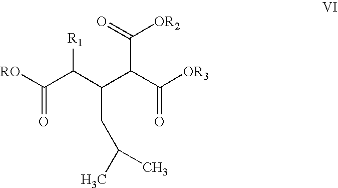
; and (c) hydrolyzing the compound of formula VI to obtain 3-isobutylglutaric acid, wherein R is H, linear or branched C₁₋₈ alkyl, or C₆₋₁₄ aryl; R₁ is H, CN, COOH, COO C₁₋₁₈ alkyl, COOC₆₋₁₄ aryl, or (R₆O)₂P═O; R₂ and R₃ are independently H, linear or branched C₁₋₈ alkyl, or C₆₋₁₄ aryl; and R₆ is linear or branched C₁₋₈ alkyl or C₆₋₁₄ aryl.

In another embodiment, the invention encompasses the 3-isobutylglutaric acid intermediate compound of the following formula IX 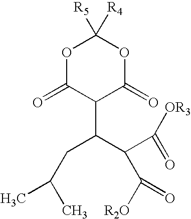
wherein R₂ and R₃ are independently H, linear or branched C₁₋₈ alkyl, or C₆₋₁₄ aryl; and R₄ and R₅ are independently H, linear or branched C₁₋₈ alkyl, or C₆₋₁₄ aryl.

In another embodiment, the invention encompasses the preparation of 3-isobutylglutaric acid from the intermediate compound of formula IX.

In another embodiment, the invention encompasses the 3-isobutylglutaric acid intermediate compound of the following formula XII 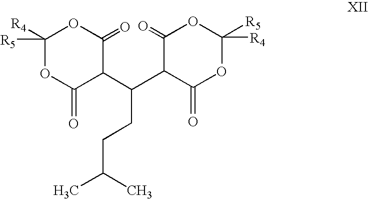
wherein R₄ and R₅ are independently H, linear or branched C₁₋₈ alkyl, or C₆₋₁₄ aryl.

In another embodiment, the invention encompasses the preparation of 3-isobutylglutaric acid from the intermediate compound of formula XII.

In another embodiment, the invention encompasses the 3-isobutylglutaric acid intermediate compound of the following formula XIII 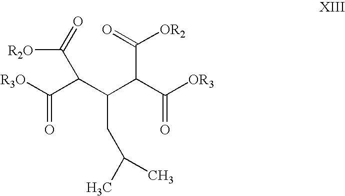
wherein R₂ and R₃ are independently H, linear or branched C₁₋₈ alkyl, or C₆₋₁₄ aryl.

In another embodiment, the invention encompasses the preparation of 3-isobutylglutaric acid from the intermediate compound of formula XIII.

In another embodiment, the invention encompasses the 3-isobutylglutaric acid intermediate compound of the following formula XIV 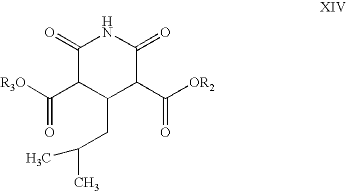
wherein R₂ and R₃ are independently H, linear or branched C₁₋₈ alkyl, or C₆₋₁₄ aryl.

In another embodiment, the invention encompasses the preparation of 3-isobutylglutaric acid from the intermediate compound of formula XIV.

### DETAILED DESCRIPTION OF THE INVENTION

The invention addresses the above-described shortcomings of the prior art by providing one-pot syntheses of the pregabalin intermediate 3-isobutylglutaric acid. These syntheses can be performed in shorter time periods than those described above, and, thus, are more feasible for use on an industrial scale.

In one embodiment, the invention encompasses a synthesis of 3-isobutylglutaric acid (denominated “Process No. 1”) that may be illustrated by the following Scheme 3. 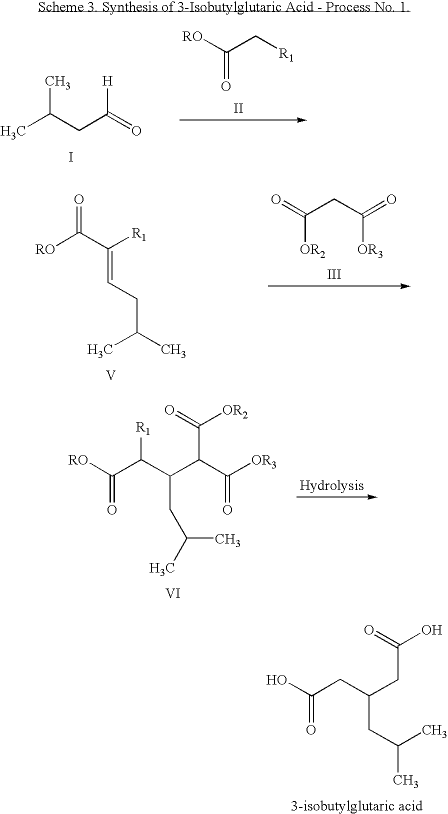
wherein R is H, linear or branched C₁₋₈ alkyl, or C₆₋₁₄ aryl; R₁ is H, CN, COOH, COO C₁₋₈ alkyl, COOC₆₋₁₄ aryl, or (R₆O)₂P═O; R₂ and R₃ are independently H, linear or branched C₁₋₈ alkyl, or C₆₋₁₄ aryl; and R₆ is linear or branched C₁₋₈ alkyl or C₆₋₁₄ aryl. Preferably, at least one of R, R₂, and R₃ is ethyl. Preferably, R₁ is cyano. Preferably, R₆ is methyl, ethyl, or phenyl.

When R is ethyl and R₁ is CN, the compound of formula II is ethylcyanoacetate of the formula, 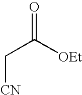
and the compound of formula V has the following structure. 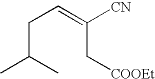
When R₂ and R₃ are ethyl, the compound of formula III is diethylmalonate of the formula. 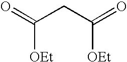
When R is ethyl, R₁ is CN, and R₂ and R₃ are ethyl, the compound of formula VI has the following structure.

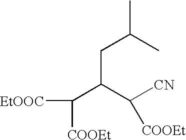

The process comprises: (a) combining isovaleraldehyde of formula I, a compound of formula II, a non-polar organic solvent, and a first base to obtain a compound of formula V; (b) combining the compound of formula V with a compound of formula III and a second base to obtain a compound of formula VI; and (c) hydrolyzing the compound of formula VI to obtain 3-isobutylglutaric acid.

The process may be performed in one-pot, i.e., without recovering the intermediates that are obtained during the process.

Preferably, the non-polar organic solvent is selected from a group consisting of linear, branched, or cyclic C₆₋₉ hydrocarbons and C₆₋₉ aromatic hydrocarbons. Preferably, the linear, branched, or cyclic C₆₋₉ hydrocarbon is hexane, heptane or cyclohexane, and more preferably cyclohexane. Preferably, the C₆₋₉ aromatic hydrocarbon is toluene. More preferably, the non-polar organic solvent is a linear, branched or cyclic C₆₋₉ hydrocarbon, and more preferably cyclohexane.

The first and the second base may be the same or different. Preferably, the first and second bases are organic or inorganic bases. Preferred organic bases are di-n-propylamine, triethylamine, piperidine, and diisopropylamine, and a more preferred organic base is di-n-propylamine. Preferred inorganic bases are potassium carbonate, cesium carbonate and sodium carbonate, and a more preferred inorganic base is potassium carbonate. More preferably, the first and second bases are organic bases, and most preferably di-n-propylamine.

Typically, the combination of step (a) is heated and water is azeotropically removed during the course of the reaction to promote the formation of the compound of formula V. Preferably, the combination of step (a) is heated to a temperature of about 20° C. to about 90° C., more preferably about 50° C. to about 90° C., and most preferably about 70° C. to about 80° C. After the water is completely removed, the non-polar organic solvent is preferably removed to obtain a concentrated mixture having the compound of formula V.

Typically, the concentrated mixture having the compound of formula V is cooled prior to combining with the compound of formula III and the second base. Preferably, the concentrated mixture is cooled to a temperature of about 35° C. to about 20° C., and more preferably about 30° C. to about 25° C.

Typically, the combination of step (b) is heated to obtain a mixture having the compound of formula VI. Preferably, the combination of step (b) is heated to a temperature of about 35° C. to about 60° C., more preferably about 40° C. to about 60° C., and most preferably about 50° C. to about 55° C. Preferably the combination is heated for about 0.5 to about 10 hours, and more preferably for about 0.5 to about 5 hours.

Typically, the mixture having the compound of formula VI is cooled prior to hydrolysis. Preferably, the mixture having the compound of formula VI is cooled to a temperature of about 50° C. to about 15° C., more preferably, about 40° C. to about 20° C., and most preferably to about 30° C. to about 25° C.

Typically, the compound of formula VI is hydrolyzed by combining with an acid and heating. Preferably, the acid is a mineral acid, an organic acid, or a mixture thereof. Preferably, the mineral acid is HCl, HBr, or sulfuric acid. Preferably, the organic acid is trifluoroacetic acid. More preferably, the acid is a mineral acid, even more preferably HBr, HCl or sulfuric acid, and most preferably, either HBr or HCl. Preferably, the acid is in the form of an aqueous solution.

Preferably, the combination of the compound of formula VI and the acid is heated to a temperature of about 80° C. to about 140° C. to obtain the 3-isobutylglutaric acid, more preferably about 90° C. to about 130° C., and most preferably about 100° C. to about 125° C. When the acid is HBr, preferably, the combination is heated for about 6 to about 20 hours, more preferably for about 6 to about 16 hours, depending on the amount of acid that is used.

The 3-isobutylglutaric acid thus obtained may be recovered by cooling the resulting biphasic mixture to a temperature of about 30° C. to about 25° C., extracting the 3-isobutylglutaric acid from the mixture with toluene, and removing the toluene to recover the 3-isobutylglutaric acid. Preferably, the toluene is removed by distillation.

Optionally, Process No. 1 may be performed in two steps instead of three, i.e., the isovaleraldehyde of formula I, the compound of formula II, and the compound of formula III may be combined in a single step. The process comprises: (a) combining isovaleraldehyde of formula I, a compound of formula II, a compound of formula III, a non-polar organic solvent, and a base to obtain a compound of formula VI; and (b) hydrolyzing the compound of formula VI to obtain 3-isobutylglutaric acid.

Preferably, the non-polar organic solvent, the base, and hydrolysis conditions are as described above.

Typically, the compound of formula I, the compound of formula II, and the compound of formula III are combined with a base, and with a non-polar organic solvent to obtain a first mixture. The first mixture is then heated and water is azeotropically removed during the course of the reaction. Preferably, the first mixture is heated to a temperature of about 40° C. to about 90° C., and more preferably about 40° C. to about 45° C. After the water is completely removed, an additional amount of base is preferably added to form a second mixture. The second mixture is then heated to obtain the compound of formula VI. Preferably, the second mixture is heated to a temperature of about 35° C. to about 60° C., more preferably about 40° C. to about 60° C., and most preferably about 50° C. to about 55° C. Preferably, the second mixture is heated for about 0.5 to about 6 hours, and more preferably for about 2 to about 5 hours. Preferably, the non-polar organic solvent is removed during heating to provide a concentrated second mixture. The concentrated second mixture is then cooled prior hydrolysis. Preferably, the second mixture is cooled to a temperature of about 35° C. to about 0° C., and more preferably about 35° C. to about 30° C.

The hydrolysis is typically performed by combining the concentrated second mixture with an acid and heating. The combination is preferably heated to a temperature of about 80° C. to about 140° C., more preferably about 90° C. to about 130° C., and most preferably about 100° C. to about 125° C. Preferably, the combination is heated for about 2 to about 20 hours, more preferably for about 6 to about 20 hours, and most preferably for about 6 to about 10 hours in the case of HBr.

In another embodiment, the invention encompasses syntheses of 3-isobutylglutaric acid (collectively denominated “Process No. 2”) that may be illustrated by each of the three processes depicted in the following Scheme 4. 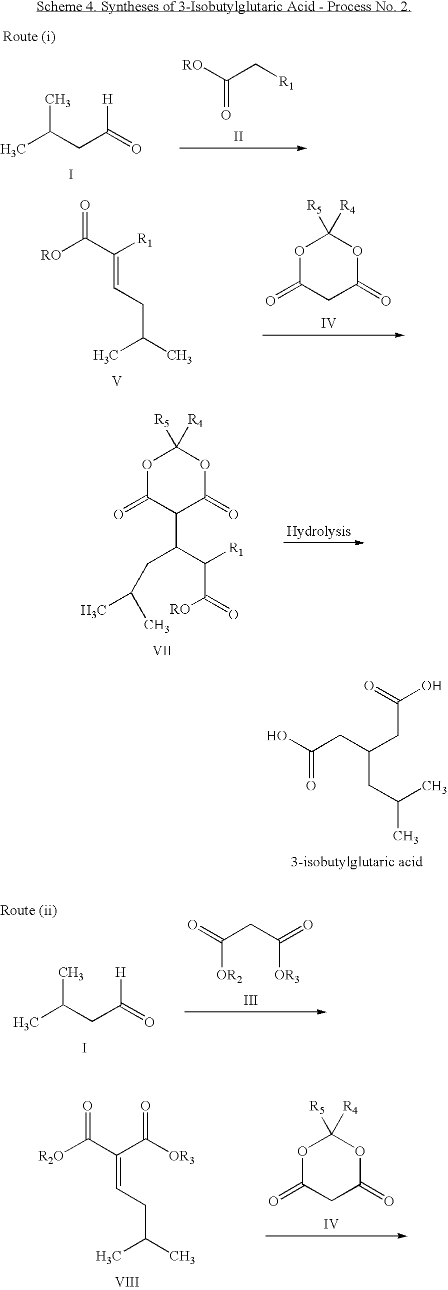 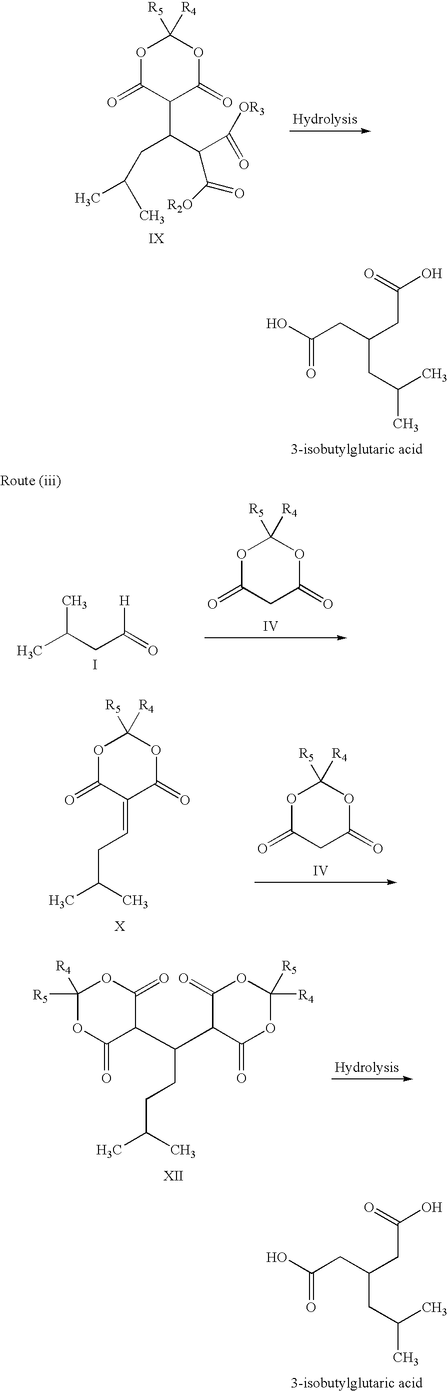
wherein R is H, linear or branched C₁₋₈ alkyl, or C₆₋₁₄ aryl; R₁ is H, CN, COOH, COO C₁₋₈ alkyl, COOC₆₋₁₄ aryl, or (R₆O)₂P═O; R₂ and R₃ are independently H, linear or branched C₁₋₈ alkyl, or C₆₋₁₄ aryl; R₄ and R₅ are independently H, linear or branched C₁₋₈alkyl, or C₆₋₁₄ aryl; and R₆ is linear or branched C₁₋₈ alkyl or C₆₋₁₄ aryl. Preferably, at least one of R, R₂, and R₃ is ethyl. Preferably, R₁ is cyano. Preferably, at least one of R₄ and R₅ is methyl. Preferably, R₆ is methyl, ethyl, or phenyl.

When R₄ and R₅ are methyl, the compound of formula IV is 2,2-dimethyl-1,3-dioxane-4,6-dione of the formula. 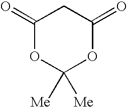
When R is Et, R₁ is CN, and R₄ and R₅ are methyl, the compound of formula VII has the following structure. 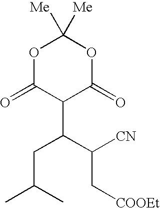
When R₂ and R₃ are ethyl, the compound of formula VIII has the following structure. 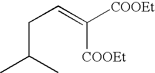
When R₄ and R₅ are methyl, the compound of formula X has the following structure. 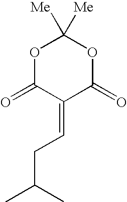
When R₄ and R₅ are methyl, the compound of formula XI has the following structure.

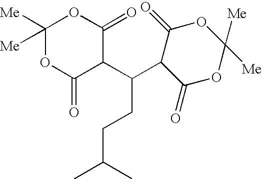

The process comprises: (a) combining isovaleraldehyde of formula I, a compound of formula II, a compound of formula III, or a compound of formula IV, a non-polar organic solvent, and a first base to obtain a compound of formula V, a compound of formula VIII, or a compound of formula X, respectively; (b) combining a compound of formula IV with the compound of formula V, the compound of formula VIII, or the compound of formula X, a polar aprotic organic solvent, and a second base to obtain a compound of formula VII, a compound of formula IX, or a compound of formula XII, respectively; and (c) hydrolyzing the compound of formula VII, the compound of formula IX, or the compound of formula XII to obtain 3-isobutylglutaric acid.

The process may be performed in one-pot, i.e., without recovering the intermediates that are obtained during the process.

Preferably, the non-polar organic solvent is selected from a group consisting of linear, branched, or cyclic C₆₋₉ hydrocarbons and C₆₋₉ aromatic hydrocarbons. Preferably, the linear, branched, or cyclic C₆₋₉ hydrocarbon is hexane, heptane or cyclohexane, and more preferably cyclohexane. Preferably, the C₆₋₉ aromatic hydrocarbon is toluene. More preferably, the non-polar organic solvent is a linear, branched or cyclic C₆₋₉ hydrocarbon, and more preferably cyclohexane.

The first and the second base may be the same or different, and preferably are different. Preferably, the first and second bases are organic or inorganic bases. Preferred organic bases are di-n-propylamine, triethylamine, piperidine, and diisopropylamine, and a more preferred organic base is di-n-propylamine. Preferred inorganic bases are potassium carbonate, cesium carbonate and sodium carbonate, and a more preferred inorganic base is potassium carbonate. More preferably, the first base is an organic base, and most preferably di-n-propylamine. More preferably, the second base is an inorganic base, and most preferably potassium carbonate.

Preferably, the polar aprotic organic solvent is dimethylsulfoxide (“DMSO”), N-N-dimethylformamide (“DMF”), or dimethylacetamide (“DMA”). More preferably, the polar aprotic organic solvent is DMSO.

Typically, the combination of step (a) is heated and water is azeotropically removed during the course of the reaction to promote the formation of the compound of formula V, the compound of formula VIII, or the compound of formula X. Preferably, the combination of step (a) is heated to a temperature of about 40° C. to about 90° C., more preferably about 50° C. to about 90° C., and most preferably about 70° C. to about 80° C. After the water is completely removed, the non-polar organic solvent is preferably removed to obtain a concentrated mixture having the compound of formula V, the compound of formula VIII, or the compound of formula X.

Typically, the concentrated mixture having the compound of formula V, the compound of formula VIII, or the compound of formula X is cooled prior to combining with the polar aprotic organic solvent and the second base. Preferably, the concentrated mixture is cooled to a temperature of about 35° C. to about 20° C., and more preferably to about 30° C. to about 25° C.

Typically, the combination of step (b) is heated to obtain the compound of formula VII, the compound of formula IX, or the compound of formula XII. Preferably, the combination of step (b) is heated to a temperature of about 35° C. to about 60° C., more preferably about 40° C. to about 60° C., and most preferably about 50° C. to about 55° C. Preferably the combination is heated for about 0.5 to about 10 hours, and more preferably about 0.5 to about 5 hours.

Typically, the compound of formula VII, the compound of formula IX or the compound of formula XII is hydrolyzed by combining with an acid and heating. Preferably, the acid is a mineral acid, an organic acid, or a mixture thereof. Preferably, the mineral acid is HCl, HBr, or sulfuric acid. Preferably, the organic acid is trifluoroacetic acid. More preferably, the acid is a mineral acid, even more preferably HBr, HCl or sulfuric acid, and most preferably, either HBr or HCl. Preferably, the acid is in the form of an aqueous solution.

Preferably, the combination of the compound of formula VII, the compound of formula IX or the compound of formula XII and the acid is heated to a temperature of about 80° C. to about 140° C. to obtain the 3-isobutylglutaric acid, more preferably about 90° C. to about 130° C., and most preferably about 100° C. to about 125° C. Preferably, the combination is heated for about 12 to about 24 hours, more preferably for about 12 to about 15 hours.

The 3-isobutylglutaric acid thus obtained may be recovered by cooling the resulting biphasic mixture to a temperature of about 30° C. to about 25° C., extracting the 3-isobutylglutaric acid from the mixture with toluene, and removing the toluene to recover the 3-isobutylglutaric acid. Preferably, the toluene is removed by distillation.

The invention further encompasses the 3-isobutylglutaric acid intermediate compound of the following formula IX 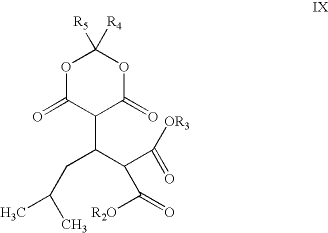
wherein R₂ and R₃ are independently H, linear or branched C₁₋₈ alkyl, or C₆₋₁₄ aryl; and R₄ and R₅ are independently H, linear or branched C₁₋₈ alkyl, or C₆₋₁₄ aryl. Preferably, at least one of R₂ and R₃ is ethyl, and at least one of R₄ and R₅ is methyl. When R₂ and R₃ are ethyl, and R₄ and R₅ are methyl, the compound of formula IX has the following structure.

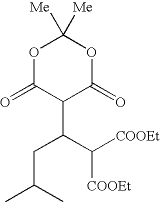

The invention further encompasses the 3-isobutylglutaric acid intermediate compound of the following formula XII 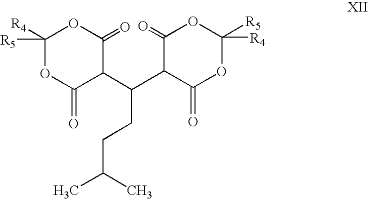
wherein R₄ and R₅ are independently H, linear or branched C₁₋₈ alkyl, or C₆₋₁₄ aryl. Preferably, at least one of R₄ and R₅ is methyl. When R₄ and R₅ are methyl, the compound of formula XII has the following structure.

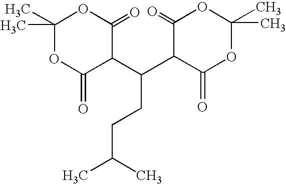

In another embodiment, the invention encompasses a synthesis of 3-isobutylglutaric acid (denominated “Process No. 3”) that may be illustrated by the following Scheme 5. 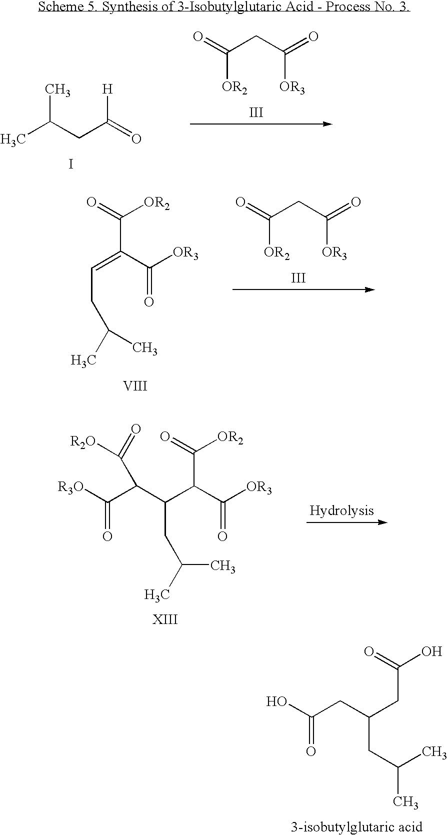
wherein R₂ and R₃ are independently H, linear or branched C₁₋₈ alkyl, or C₆₋₁₄ aryl. Preferably, at least one of R₂ and R₃ is ethyl.

The process comprises: (a) combining isovaleraldehyde of formula I, a compound of formula III, a non-polar organic solvent, an organic acid, and an organic base to obtain a compound of formula VIII; (b) combining the compound of formula VIII with a compound of formula III, a polar aprotic organic solvent, and an inorganic base to obtain a compound of formula XIII; and (c) hydrolyzing the compound of formula XIII to obtain 3-isobutylglutaric acid.

The process may be performed in one-pot, i.e., without recovering the intermediates that are obtained during the process.

Preferably, the non-polar organic solvent is selected from linear, branched, or cyclic C₆₋₉ hydrocarbons and C₆₋₉ aromatic hydrocarbons. Preferably, the linear, branched, or cyclic C₆₋₉ hydrocarbon is hexane, heptane or cyclohexane, and more preferably cyclohexane. Preferably, the C₆₋₉ aromatic hydrocarbon is toluene. More preferably, the non-polar organic solvent is a linear, branched or cyclic C₆₋₉ hydrocarbon, and more preferably cyclohexane.

Preferably, the organic base is di-n-propylamine, triethylamine, piperidine, or diisopropylamine, and more preferably di-n-propylamine.

Preferably, the inorganic base is potassium carbonate, cesium carbonate or sodium carbonate, and more preferably potassium carbonate.

Preferably, the polar aprotic organic solvent is dimethylsulfoxide (“DMSO”), N—N-dimethylformamide (“DMF”), or dimethylacetamide (“DMA”). More preferably, the polar aprotic organic solvent is DMSO.

Typically, the combination of step (a) is heated and water is azeotropically removed during the course of the reaction to promote the formulation of the compound of formula VIII. Preferably, the combination of step (a) is heated to a temperature of about 20° C. to about 90° C., more preferably about 40° C. to about 90° C., even more preferably about 50° C. to about 90° C., and most preferably about 70° C. to about 80° C.

Typically, the concentrated mixture having the compound of formula VIII is cooled prior to combining with the polar aprotic organic solvent, the compound of formula III, and the inorganic base. Preferably, the concentrated mixture is cooled to a temperature of about 35° C. to about 20° C., and more preferably about 30° C. to about 25° C.

Typically, the combination of step (b) is heated to obtain a mixture having the compound of formula XIII. Preferably, the combination of step (b) is heated to a temperature of about 20° C. to about 45° C., and more preferably about 25° C. to about 30° C. Preferably, the combination is heated for about 2 to about 10 hours, and more preferably about 4 to about 6 hours.

Optionally, the process may further comprise, prior to hydrolysis: (a) cooling the mixture having the compound of formula XIII; (b) combining the mixture having the compound of formula XIII with an alcohol and sodium hydroxide to obtain a mixture having a basic pH; (c) cooling the mixture having the basic pH; (d) combining the mixture having the basic pH with glacial acetic acid and HCl to obtain a mixture having an acidic pH; and (e) removing the alcohol.

Preferably, the mixture having the compound of formula XIII is cooled to a temperature of about −5° C. to about −20° C., and more preferably about −5° C. to about −10° C. Preferably, the basic pH is about 7 to about 10 and more preferably about 8. Preferably, the mixture having the basic pH is cooled for about 1 to about 5 hours, and more preferably about 2 to about 3 hours. Preferably, the acidic pH is about 3 to about 6, and more preferably about 5 to about 6. Preferably, the alcohol is a C₁₋₄ alcohol. More preferably, the C₁₋₄ alcohol is methanol, ethanol, isopropanol or butanol, more preferably, ethanol.

Typically, the compound of formula XIII is hydrolyzed by combining with an acid and heating. Preferably, the acid is a mineral acid, an organic acid, or a mixture thereof. Preferably, the mineral acid is HCl, HBr, or sulfuric acid. Preferably, the organic acid is trifluoroacetic acid, acetic acid, formic acid, or propionic acid. More preferably, the acid is a mineral acid, even more preferably HBr, HCl or sulfuric acid, and most preferably, either HBr or HCl. Preferably, the acid is in the form of an aqueous solution. More preferably, the organic acid is acetic acid.

Preferably, the combination the compound of formula XIII and the acid is heated to a temperature of about 80° C. to about 140° C. to obtain the 3-isobutylglutaric acid, more preferably about 90° C. to about 130° C., and most preferably about 100° C. to about 125° C. Preferably, the combination is heated for about 12 to about 24 hours, more preferably for about 20 to about 24 hours.

The 3-isobutylglutaric acid thus obtained may be recovered by cooling the resulting biphasic mixture to a temperature of about 30° C. to about 25° C., extracting the 3-isobutylglutaric acid from the mixture with toluene, and removing the toluene to recover the 3-isobutylglutaric acid. Preferably, the toluene is removed by distillation.

Optionally, process No. 3 may be done in two steps instead of three, i.e., the isovaleraldehyde can be reacted with about two mole equivalents of the compound of formula III in a single step. The process comprises (a) combining isovaleraldehyde of formula I, a compound of formula III, an alcohol, ammonium acetate and ammonia to obtain a compound of formula XIV; and (b) hydrolyzing the compound of formula XIV to obtain 3-isobutylglutaric acid. The process may be illustrated by the following Scheme 6. 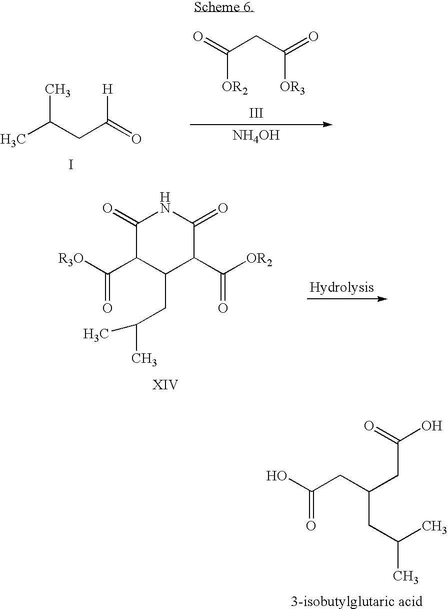
wherein R₂ and R₃ are independently H, linear or branched C₁₋₈ alkyl, or C₆₋₁₄ aryl. Preferably, at least one of R₂ and R₃ is ethyl.

Preferably, the compound of formula III is combined with an alcohol, ammonium acetate, the compound of formula I, and ammonia, at a temperature of about 5° C. to about 20° C., more preferably about 8° C. to about 10° C., to provide a reaction mixture. Preferably, the reaction mixture is then maintained for about 30 to about 35 minutes. The reaction mixture is then maintained at this temperature for about 20 to about 60 minutes, preferably about 30 to 35 minutes, followed by warming to a temperature of about 20° C. to about 40° C. for about 20 to about 24 hours. Preferably, the reaction mixture is warmed to a temperature of about 25° C. to about 30° C. Then, the alcohol is removed, and an acid is added followed by heating to a temperature of about 80° C. to about 140° C. for about 2 to about 12 hours, preferably about 10 to about 12 hours.

Preferably, the alcohol is a C₁₋₄ alcohol. More preferably, the C₁₋₄ alcohol is methanol, ethanol, isopropanol or butanol, and more preferably methanol.

Typically, the compound of formula XIV is hydrolyzed by combining with an acid and heating. Preferably, the acid is a mineral acid, an organic acid, or a mixture thereof. Preferably, the mineral acid is HCl, HBr, or sulfuric acid. Preferably, the organic acid is trifluoroacetic acid. More preferably, the acid is a mineral acid, even more preferably HBr, HCl or sulfuric acid, and most preferably, either HBr or HCl. Preferably, the acid is in the form of an aqueous solution.

Preferably, the combination of the compound of formula XIV and the acid is heated to a temperature of about 80° C. to about 140° C. to obtain the 3-isobutylglutaric acid, more preferably about 90° C. to about 130° C., and most preferably about 100° C. to about 125° C. Preferably, the combination is heated for about 6 to about 20 hours, more preferably for about 6 to about 16 hours, depending on the amount of acid that is used.

The 3-isobutylglutaric acid thus obtained may be recovered by the methods described above.

The 3-isobutylglutaric acid prepared by any of the above-described processes may subsequently be converted into (S)-Pregabalin. The conversion may be performed, for example, by the process disclosed in U.S. Pat. No. 5,616,793, hereby incorporated by reference.

The invention further encompasses the 3-isobutylglutaric acid intermediate compound of the following formula XIII 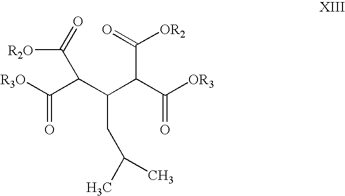
wherein R₂ and R₃ are independently H, linear or branched C₁₋₈ alkyl, or C₆₋₁₄ aryl. Preferably, at least one of R₂ and R₃ is ethyl.

The invention further encompasses the 3-isobutylglutaric acid intermediate compound of the following formula XIV 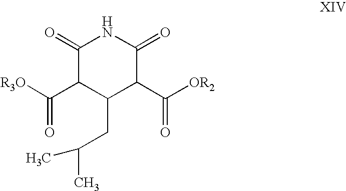
wherein R₂ and R₃ are independently H, linear or branched C₁₋₈ alkyl, or C₆₋₁₄ aryl. Preferably, at least one of R₂ and R₃ is ethyl.

Having thus described the invention with reference to particular preferred embodiments, other embodiments will become apparent to one skilled in the art from consideration of the specification. The invention is further defined by reference to the following examples describing. The Examples are set forth to aid in understanding the invention but are not intended to, and should not be construed to, limit its scope in any way. The examples do not include detailed descriptions of conventional methods. It will be apparent to those skilled in the art that many modifications, both to materials and methods may be practiced without departing from the scope of the invention.

### EXAMPLES

### Example 1

To a four neck round bottom flask fitted with a mechanical stirrer, condenser and charging tube, was charged isovaleraldehyde (1.0 kg, 11.61 mole), cyclohexane (1.35 L), ethyl cynoacetate (1.28 kg, 11.38 mole) and di-n-propylamine (11.74 g). The reaction mass was heated to reflux and water was removed azeotropically. After complete removal of water (˜208 ml), cyclohexane was distilled from the reaction mass followed by removal of traces of cyclohexane under vacuum. The reaction mass was cooled to 30-35° C. and diethyl malonate (2.027 kg, 12.67 mole) was added followed by addition of di-n-propylamine (106.91 g). The reaction mass was heated to 50-55° C. for 3-5 hours and then cooled to 25-30° C. Then hydrobromic acid (47%, 23.76 L) was added and the mass was refluxed at 100-125° C. for 6-10 hours. The reaction mass was cooled to 25-30° C. and extracted with toluene. The toluene was distilled off to obtain 3-isobutylglutaric acid in a yield of 1.54 kg (71%) having GC purity of 93.59%.

### Example 2

To a four neck round bottom flask fitted with a mechanical stirrer, condenser, and charging tube was charged isovaleraldehyde (1.0 kg, 11.61 mole), cyclohexane (1.35 L), ethyl cynoacetate (1.28 kg, 11.38 mole) and di-n-propylamine (11.74 g). The reaction mass was heated to reflux and water was removed azeotropically. After complete removal of water (˜208 ml), cyclohexane was distilled from the reaction mass followed by removal of traces of cyclohexane under vacuum. The reaction mass was cooled to 30-35° C. and diethyl malonate (2.027 kg, 12.67 mole) was added followed by addition of di-n-propylamine (106.91 g). The reaction mass was heated to 50-55° C. for 3-5 hours and then cooled to 25-30° C. Then an aqueous solution of hydrochloric acid (35% hydrochloric acid, 19.79 L in 3.95 L of water) was added and the mass was refluxed at 100-125° C. for 50-100 hours. The reaction mass was cooled to 25-30° C. and extracted with toluene. The toluene was distilled off to obtain 3-isobutylglutaric acid in a yield of 1.66 kg (76.14%) having GC purity of 93.64%.

### Example 3

To a four neck round bottom flask fitted with a mechanical stirrer, condenser and charging tube, was charged isovaleraldehyde (25 g, 0.290 mole), cyclohexane (34 ml), ethyl cynoacetate (32.19 g 0.284 mole) and di-n-propylamine (0.29 g). The reaction mass was heated to reflux and water was removed azeotropically. After complete removal of water, cyclohexane was distilled from the reaction mass followed by removal of traces of cyclohexane under vacuum. The reaction mass was cooled to 30-35° C. and was charged with dimethylsulfoxide (20 ml) followed by addition of 2,2-dimethyl-1,3-dioxane-4,6-dione (41.86 g, 0.290 mole) and potassium carbonate (19 g, 0.137 mole). The reaction mass was stirred for 1-2 hours at 45-50° C. Then, hydrochloric acid (6N, 300 ml) was added and the mass was refluxed at a temperature 100-125° C. for 15-20 hours. The reaction mass was cooled to 25-30° C. and was extracted with dichloromethane. The dichloromethane was distilled off to obtain 3-isobutylglutaric acid in a yield of 34.4 g (66%) having GC purity 70.88%.

### Example 4

To a four neck round bottom flask fitted with a mechanical stirrer, condenser and charging tube, was loaded isovaleraldehyde (10 g, 0.16 mole), cyclohexane (13.6 ml), 2,2-dimethyl-1,3-dioxane-4,6-dione (22.57 g, 0.156 mole) and di-n-propylamine (0.2 ml). The reaction mass was heated to reflux and water was removed azeotropically. After complete removal of water, cyclohexane was distilled from the reaction mass followed by removal of traces of cyclohexane under vacuum. The reaction mass was cooled to 30-35° C. and was charged with dimethylsulfoxide (13 ml) followed by addition of 2,2-dimethyl-1,3-dioxane-4,6-dione (22.57 g, 0.156 mole) and potassium carbonate (10.76 g). The reaction mass was stirred for 1-2 hours at 45-50° C. to obtain the compound of formula X1. Then HCl was added (6N, 300 ml) and the mass was refluxed at temperature of 100-125° C. for 12-15 hours. The reaction mass was cooled to 25-30° C. and was extracted with dichloromethane. The dichloromethane was distilled off to obtain 3-isobutylglutaric acid in a yield of 14.32 g (65.5%) having GC purity of 75%.

### Example 5

To a four neck round bottom flask fitted with a mechanical stirrer, condenser and charging tube, was loaded isovaleraldehyde (25 g, 0.290 mole), cyclohexane (34 ml), diethyl malonate (45.58 g, 0.285 mole) and di-n-propylamine (0.4 ml). The reaction mass was heated to reflux and water was removed azeotropically. After complete removal of water, cyclohexane was distilled off from the reaction mass, followed by removal of traces of cyclohexane under vacuum. The reaction mass was cooled to 30-35° C., and was charged with dimethylsulfoxide (13 ml), followed by addition of 2,2-dimethyl-1,3-dioxane-4,6-dione (41.46 g, 0.288 mole) and potassium carbonate (23.72 g). The reaction mass was stirred for 1-2 hours at 45-50° C. to obtain the compound of formula IX. The HCl was added (6N, 650 ml) and the mass was refluxed at temperature of 100-125° C. for 12-15 hours. The reaction mass was cooled to 25-30° C. and was extracted with dichloromethane. The dichloromethane was distilled off to get 3-isobutylglutaric acid in a yield of 34.4 g (63.1%) having GC purity of 90.7%.

### Example 6

To a four neck round bottom flask fitted with a mechanical stirrer, condenser and charging tube, was loaded isovaleraldehyde (18.85 g, 0.219 mole), cyclohexane (52 ml), glacial acetic acid (1.24 g), diethyl malonate (32.32 g, 0.202 mole) and di-n-propylamine (1.04 g). The reaction mass was heated to reflux and water was removed azeotropically. After complete removal of water, cyclohexane was distilled off from the reaction mass, followed by removal of traces of cyclohexane under vacuum. The reaction mass was cooled to 30-35° C. and was charged with dimethylsulfoxide (25 ml), followed by addition of diethyl malonate (35.0 g, 0.219 mole) and potassium carbonate (16.67 g). The reaction mass was stirred for 4-6 hours at 25-30° C. to obtain the compound of formula IV. Then, HCl (6N, 400 ml) was added and the mass was refluxed at temperature of 100-125° C. for 20-24 hours. The reaction mass was cooled to 25-30° C. and extracted with toluene. The toluene was distilled off to obtain 3-isobutylglutaric acid in a yield of 27.8 g (67.57%) having GC purity of 93.81%.

### Example 7

To a four neck round bottom flask fitted with a mechanical stirrer, condenser and charging tube, was loaded isovaleraldehyde (18.85 g, 0.218 mole), cyclohexane (52 ml), diethyl malonate (33.32 g 0.208 mole), glacial acetic acid (1.24 g) and di-n-propylamine (1.04 g). The reaction mass was heated to reflux and water was removed azeotropically. After complete removal of water, cyclohexane was distilled from the reaction mass followed by removal of traces of cyclohexane under vacuum. The reaction mass was cooled to 30-35° C. and was charged with dimethylsulfoxide (25 ml) followed by addition of diethyl malonate (35 g, 0.218 mole) and potassium carbonate (16.67 g, 0.12 mole) and was stirred for 3-4 hours at 25-30° C. to obtain the compound of formula IV. The mass was cooled to −5 to −10° C. followed by addition of ethanol (100 ml) and sodium hydroxide solution to raise the pH to alkaline pH. The mass was stirred for 2-3 hours at −5° to −10° C. The pH of the reaction mass was lowered to 5-6 using glacial acetic acid/hydrochloric acid and then, ethanol was distilled out. After the removal of ethanol, hydrochloric acid (35%, 1.0 L) was added and the mass was refluxed at temperature of 100-125° C. for 20-24 hours. The reaction mass was cooled to 25-30° C. and was extracted with toluene. The toluene was distilled off to obtain 3-isobutylglutaric acid in a yield of 30 g (72.9%) having GC purity 96.4%.

### Example 8

To a four neck round bottom flask fitted with a mechanical stirrer, condenser and charging tube, was charged diethyl malonate (232.8 g, 1.45 mole), and methanol (50 ml). The reaction mass was cooed to 8-10° C., followed by addition of isovaleraldehyde (50 g, 0.58 mole), ammonium acetate (4 g) and aqueous ammonia (25%, 99 g) at 8-10° C. The reaction mass was stirred at 8-10° C. for 30-35 minutes, followed by stirring at 25-30° C. for 20-24 hours. Then, methanol was distilled off followed by addition of 6N hydrochloric acid (1.5 L). The mass was refluxed at a temperature of 110-115° C. for 10-12 hours. The reaction mass was cooled to 25-30° C. and was extracted with toluene. The toluene was distilled off to obtain 3-isobutylglutaric acid in a yield of 27.2 g (24.9%) having GC purity of 60.5%.

### Example 9

To a four neck round bottom flask fitted with a mechanical stirrer, condenser and charging tube, was charged isovaleraldehyde (50 g, 0.58 mole), cyclohexane. (67.5 ml), ethyl cynoacetate (64.4 g 0.57 mole), and diethyl malonate (100.4 g 0.62 mole) and di-n-propylamine (0.76 ml). The reaction mass was heated to 40-45° C., and water was separated, and di-n-propylamine (4.0 ml 0.029 mole) was further added. The reaction mass was heated to 50-55° C. for 2-5 hours, and cyclohexane was distilled from the reaction mass followed by removal of traces of cyclohexane under vacuum. The reaction mass was cooled to 30-35° C. and then hydrobromic acid (47%, 800 ml) was added and the mass was refluxed at 100-125° C. for 6-10 hours. The reaction mass was cooled to 25°-30° C. and extracted with toluene. The toluene was distilled off to get 3-isobutylglutaric acid in a yield of 77.2 g (70%) having GC purity of 96.06%.

### Example 10

To a four neck round bottom flask fitted with a mechanical stirrer, condenser, and charging tube was charged isovaleraldehyde (1.0 kg, 11.61 mole), cyclohexane (1.35 L), ethyl cynoacetate (1.28 kg, 11.38 mole) and di-n-propylamine (11.74 g). The reaction mass was heated to reflux and water was removed azeotropically. After complete removal of water (˜208 ml), cyclohexane was distilled from the reaction mass followed by removal of traces of cyclohexane under vacuum. The reaction mass was cooled to 30-35° C. and diethyl malonate (2.027 kg, 12.67 mole) was added followed by addition of di-n-propylamine (106.91 g). The reaction mass was heated to 50-55° C. for 3-5 hours and then cooled to 25-30° C. Then an aqueous solution of hydrochloric acid (35% hydrochloric acid, 19.79 L in 3.95 L of water) was added and the mass was refluxed at 100-125° C. for 20-25 hours. A portion of low boiling material was allowed to distill out followed by addition of aqueous solution of hydrochloric acid (35% hydrochloric acid, 1-1.25 L in 1-1.25 L of water). The mass was refuxed for 50-100 h. The reaction mass was cooled to 25-30° C. and extracted with toluene. The toluene was distilled off to obtain 3-isobutylglutaric acid in a yield of 1.7 kg (77.9%) having GC purity of 95.2%.

We claim:

1. A compound of the following formula IX 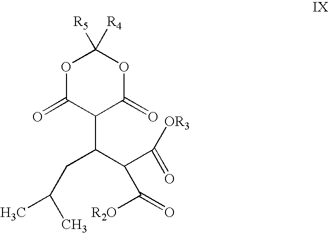
wherein R₂ and R₃ are independently H, linear or branched C₁₋₈ alkyl, or C₆₋₁₄ aryl and R₄ and R₅ are independently H, linear or branched C₁₋₈ alkyl, or C₆₋₁₄ aryl.

2. The compound of claim 1, wherein at least one of R₂ and R₃ is ethyl and at least one of R₄ and R₅ is methyl.

3. A compound of the following formula XII 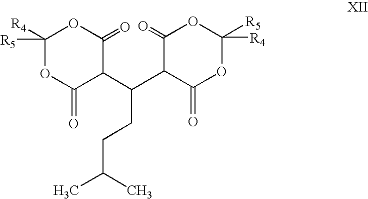
wherein R₄ and R₅ are independently H, linear or branched C₁₋₈ alkyl, or C₆₋₁₄ aryl.

4. The compound of claim 3, wherein at least one of R₄ and R₅ is methyl.

5. A compound of the following formula XIII 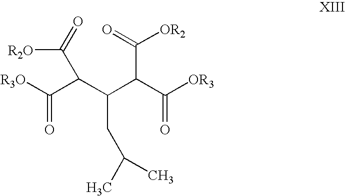
wherein R₂ and R₃ are independently H, linear or branched C₁₋₈ alkyl, or C₆₋₁₄ aryl.

6. The compound of claim 5, wherein at least one of R₂ and R₃ is ethyl.

7. A compound of the following formula XIV 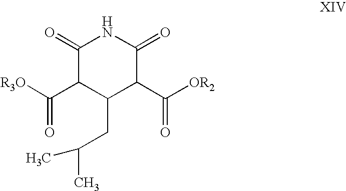
wherein R₂ and R₃ are independently H, linear or branched C₁₋₈ alkyl, or C₆₋₁₄ aryl.

8. The compound of claim 7, wherein at least one of R₂ and R₃ is ethyl.

9. A process for preparing 3-isobutylglutaric acid comprising:
(a) combining isovaleraldehyde, a compound of the following formula II, 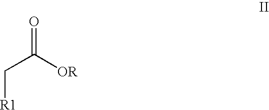
a compound of the following formula III, 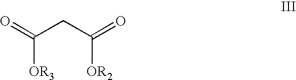
or a compound of the following formula IV, 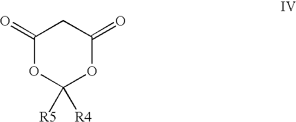
a non-polar organic solvent, and a first base to obtain a compound of the following formula V, 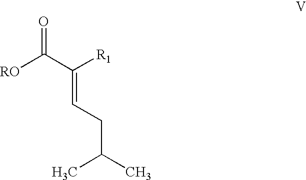
a compound of the following formula VIII, 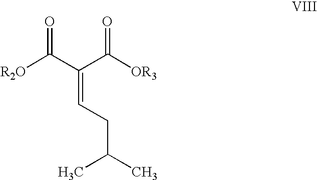
or a compound of the following formula X, 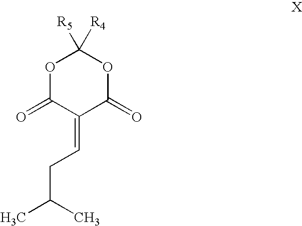
respectively;
(b) combining a compound of formula IV with the compound of formula V, the compound of formula VIII, or the compound of formula X, a polar aprotic organic solvent, and a second base to obtain a compound of the following formula VII, 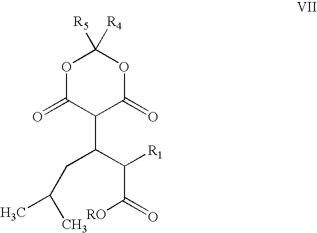
a compound of the following formula IX, 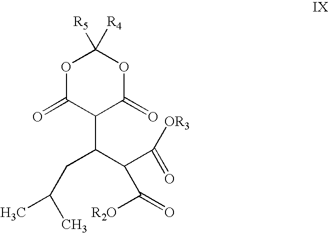
or a compound of the following formula XII, 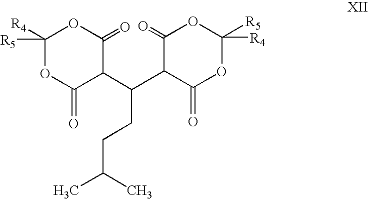
respectively; and
(c) hydrolyzing the compound of formula VII, the compound of formula IX, or the compound of formula XII to obtain 3-isobutylglutaric acid,
wherein R is H, linear or branched C₁₋₈ alkyl, or C₆₋₁₄ aryl; R₁ is H, CN, COOH, COO C₁₋₈ alkyl, COOC₆₋₁₄ aryl, or (R₆O)₂P═O; R₂ and R₃ are independently H, linear or branched C₁₋₈ alkyl, or C₆₋₁₄ aryl; R₄ and R₅ are independently H, linear or branched C₁₋₈ alkyl, or C₆₋₁₄ aryl; and R₆ is linear or branched C₁₋₈ alkyl or C₆₋₁₄ aryl.

10. The process of claim 9, wherein the process is a one-pot process.

11. The process of claim 9, wherein at least one of R, R₂, and R₃ is ethyl.

12. The process of claim 9, wherein R₁ is cyano.

13. The process of claim 9, wherein at least one of R₄ and R₅ is methyl.

14. The process of claim 9, wherein R₆ is methyl, ethyl, or phenyl.

15. The process of claim 9, wherein the non-polar organic solvent is selected from linear, branched, or cyclic C₆₋₉ hydrocarbons and C₆₋₉ aromatic hydrocarbons.

16. The process of claim 15, wherein the linear, branched, or cyclic C₆₋₉ hydrocarbon is hexane, heptane or cyclohexane.

17. The process of claim 15, wherein the C₆₋₉ aromatic hydrocarbon is toluene.

18. The process of claim 9, wherein the first base is an organic base.

19. The process of claim 18, wherein the organic base is di-n-propylamine, triethylamine, piperidine, or diisopropylamine.

20. The process of claim 9, wherein the second base is an inorganic base.

21. The process of claim 20, wherein the inorganic base is potassium carbonate, cesium carbonate or sodium carbonate.

22. The process of claim 9, wherein the polar aprotic organic solvent is dimethylsulfoxide, N—N-dimethylformamide, or dimethylacetamide.

23. The process of claim 9, wherein the combination of step (a) is heated and water is azeotropically removed during the course of the reaction to promote the formation of the compound of formula V, the compound of formula VIII, or the compound of formula X.

24. The process of claim 23, wherein, after removal of the water, the non-polar organic solvent is removed to obtain a concentrated mixture having the compound of formula V, the compound of formula VIII, or the compound of formula X.

25. The process of claim 24, wherein the concentrated mixture is cooled prior to combining with the polar aprotic organic solvent and the second base.

26. The process of claim 9, wherein the combination of step (b) is heated to obtain the compound of formula VII, the compound of formula IX, or the compound of formula XII.

27. The process of claim 9, wherein the compound of formula VII, the compound of formula IX, or the compound of formula XII is hydrolyzed by combining with an acid and heating.

28. The process of claim 27, wherein the acid is a mineral acid, an organic acid, or a mixture thereof.

29. The process of claim 28, wherein the mineral acid is HCl, HBr, or sulfuric acid.

30. The process of claim 28, wherein the organic acid is trifluoroacetic acid.

31. The process of claim 27, wherein the combination of the compound of formula VII, the compound of formula IX, or the compound of formula XII and the acid is heated to a temperature of about 80° C. to about 140° C.

32. A process for preparing (S)-pregabalin comprising:
(a) preparing 3-isobutylglutaric acid by the process of claim 9; and
(b) converting the 3-isobutylglutaric acid into (S)-pregabalin.

33. A process for preparing 3-isobutylglutaric acid comprising:
(a) combining isovaleraldehyde, a compound of the following formula III, 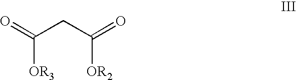
a non-polar organic solvent, an organic acid, and an organic base to obtain a compound of the following formula VIII; 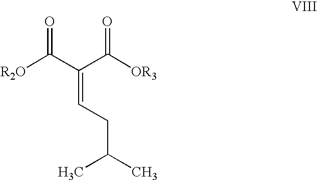
(b) combining the compound of formula VIII with the compound of formula III, a polar aprotic organic solvent, and an inorganic base to obtain a compound of the following formula XIII; and 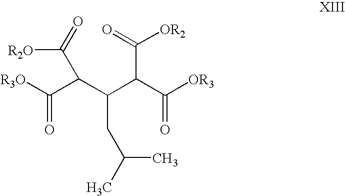
(c) hydrolyzing the compound of formula XIII to obtain 3-isobutylglutaric acid,
wherein R₂ and R₃ are independently H, linear or branched C₁₋₈ alkyl, or C₆₋₁₄ aryl.

34. The process of claim 33, wherein the process is a one-pot process.

35. The process of claim 33, wherein at least one of R₂ and R₃ is ethyl.

36. The process of claim 33, wherein the non-polar organic solvent is selected from linear, branched, or cyclic C₆₋₉ hydrocarbons and C₆₋₉ aromatic hydrocarbons.

37. The process of claim 36, wherein the linear, branched, or cyclic C₆₋₉ hydrocarbon is hexane, heptane or cyclohexane.

38. The process of claim 36, wherein the C₆₋₉ aromatic hydrocarbon is toluene.

39. The process of claim 33, wherein the organic base is di-n-propylamine, triethylamine, piperidine, or diisopropylamine.

40. The process of claim 33, wherein the inorganic base is potassium carbonate, cesium carbonate or sodium carbonate.

41. The process of claim 33, wherein the polar aprotic organic solvent is dimethylsulfoxide, N—N-dimethylformamide, or dimethylacetamide.

42. The process of claim 33, wherein the combination of step (a) is heated and water is azeotropically removed during the course of the reaction to promote the formation of the compound of formula VIII.

43. The process of claim 42, wherein, after removal of the water, the non-polar organic solvent is removed to obtain a concentrated mixture having the compound of formula VIII.

44. The process of claim 43, wherein the concentrated mixture is cooled prior to combining with the polar aprotic organic solvent, the compound of formula III and the inorganic base.

45. The process of claim 33, wherein the combination of step (b) is heated to obtain the compound of formula XIII.

46. The process of claim 33, further comprising, prior to hydrolysis,
(a) cooling the compound of formula XIII;
(b) combining the compound of formula XIII with an alcohol and sodium hydroxide to obtain a mixture having a basic pH;
(c) cooling the mixture;
(d) combining the mixture with glacial acetic acid and HCl to obtain a mixture having an acidic pH; and
(e) removing the alcohol.

47. The process of claim 46, wherein the compound of formula XIII is cooled to a temperature of about −5° C. to about −20° C.

48. The process of claim 46, wherein the basic pH is about 7 to about 10.

49. The process of claim 46, wherein the acidic pH is about 3 to about 6.

50. The process of claim 46, wherein the alcohol is a C₁₋₄ alcohol.

51. The process of claim 50, wherein the C₁₋₄ alcohol is methanol, ethanol, isopropanol or butanol.

52. The process of claim 33, wherein the compound of formula XIII is hydrolyzed by combining with an acid and heating.

53. The process of claim 52, wherein the acid is a mineral acid, an organic acid, or a mixture thereof.

54. The process of claim 53, wherein the mineral acid is HCl, HBr, or sulfuric acid.

55. The process of claim 53, wherein the organic acid is trifluoroacetic acid, acetic acid, formic acid, or propionic acid.

56. The process of claim 53, wherein the organic acid is acetic acid.

57. The process of claim 52, wherein the combination of the compound of formula XIII and the acid is heated to a temperature of about 80° C. to about 140° C.

58. A process for preparing (S)-pregabalin comprising:
(a) preparing 3-isobutylglutaric acid by the process of claim 33; and
(b) converting the 3-isobutylglutaric acid into (S)-pregabalin.

59. A process for preparing 3-isobutylglutaric acid comprising:
(a) combining isovaleraldehyde, a compound of the following formula III, 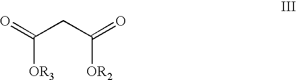
an alcohol, ammonium acetate and ammonia to obtain a compound of the following formula XIV; 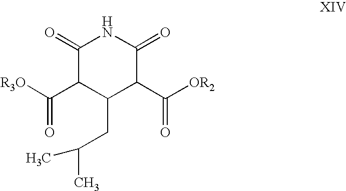
and
(b) hydrolyzing the compound of formula XIV to obtain 3-isobutylglutaric acid,
wherein R₂ and R₃ are independently H, linear or branched C₁₋₈ alkyl, or C₆₋₁₄ aryl.

60. The process of claim 59, wherein the process is a one-pot process.

61. The process of claim 59, wherein at least one of R₂ and R₃ is ethyl.

62. The process of claim 59, wherein the isovaleraldehyde, the compound of formula III, the alcohol, the ammonium acetate, and the ammonia are combined at a temperature of about 5° C. to about 20° C.

63. The process of claim 62, wherein the combination of step (a) is maintained at a temperature of about 5° C. to about 20° C. for about 20 to about 60 minutes.

64. The process of claim 62, wherein the combination of step (a) is subsequently warmed to a temperature of about 20° C. to about 40° C.

65. The process of claim 64, wherein the alcohol is removed prior to step (b).

66. The process of claim 59, wherein the alcohol is a C₁₋₄ alcohol.

67. The process of claim 59, wherein the alcohol is methanol, ethanol, isopropanol or butanol.

68. The process of claim 59, wherein the compound of formula XIV is hydrolyzed by combining with an acid and heating.

69. The process of claim 68, wherein the acid is a mineral acid, an organic acid, or a mixture thereof.

70. The process of claim 69, wherein the mineral acid is HCl, HBr, or sulfuric acid.

71. The process of claim 68, wherein the combination of the compound of formula XIV and the acid is heated to a temperature of about 80° C. to about 140° C.

72. A process for preparing (S)-pregabalin comprising:
(a) preparing 3-isobutylglutaric acid by the process of claim 59; and
(b) converting the 3-isobutylglutaric acid into (S)-pregabalin.

73. A process for preparing 3-isobutylglutaric acid comprising:
(a) combining isovaleraldehyde, a compound of the following formula II, 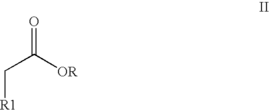
a non-polar organic solvent, and a first base to obtain a compound of the following formula V; 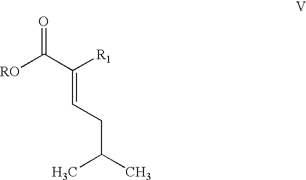
(b) combining the compound of formula V with a compound of the following formula III 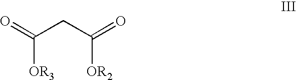
and a second base to obtain a compound of the following formula VI 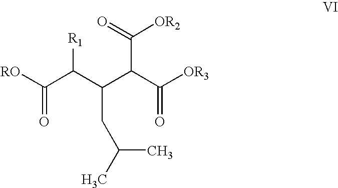
; and
(c) combining the compound of formula VI with an acid and heating to obtain 3-isobutylglutaric acid,
wherein R is H, linear or branched C₁₋₈ alkyl, or C₆₋₁₄ aryl; R₁ is H, CN, COOH, COO C₁₋₈ alkyl, COOC₆₋₁₄ aryl, or (R₆O)₂P═O; R₂ and R₃ are independently H, linear or branched C₁₋₈ alkyl, or C₆₋₁₄ aryl; and R₆ is linear or branched C₁₋₈ alkyl or C₆₋₁₄ aryl.
